(19) United States
(12) Patent Application Publication
(10) Pub. No.: US 2024/0252791 A1
(43) Pub. Date: Aug. 1, 2024

(54) STEERABLE MEDICAL DEVICES AND RELATED METHODS THEREOF

(71) Applicants: Boston Scientific Medical Device Limited, Galway (IE); Boston Scientific Scimed, Inc., Maple Grove, MN (US)

(72) Inventors: Juan Pablo ORTIZ GARCIA, Heredia (CR); Deepak Kumar SHARMA, Muzaffarnagar (IN); James J. SCUTTI, Norwell, MA (US); Sharath Kumar G., Ramanagar District (IN); Charles GIBSON, Malden, MA (US)

(73) Assignees: Boston Scientific Medical Device Limited, Galway (IE); Boston Scientific Scimed, Inc., Maple Grove, MN (US)

(21) Appl. No.: 18/422,402

(22) Filed: Jan. 25, 2024

Related U.S. Application Data
(60) Provisional application No. 63/481,615, filed on Jan. 26, 2023.

(51) Int. Cl. A61M 25/09

(52) U.S. Cl. CPC A61M 25/09; A61M 2025/09075; A61M 2025/09108; A61M 2025/09141; A61M 2025/09158

(57) ABSTRACT

A medical device comprising a handle including a handle body and an actuator, and a shaft extending distally from the handle body, wherein the shaft comprises of a shape memory material, wherein the shaft includes a proximal section and a distal section, the proximal section having a first austenitic finish temperature and the distal section having a second austenitic finish temperature, and wherein the first austenitic finish temperature is lower than the second austenitic finish temperature.

### CROSS-REFERENCE TO RELATED APPLICATIONS

This application claims priority to U.S. Provisional Application No. 63/481,615, filed on Jan. 26, 2023, which is incorporated by reference herein in its entirety.

### TECHNICAL FIELD

The disclosure relates generally to devices and methods for steerable medical devices. More specifically, aspects of the disclosure pertain to devices and/or methods for steerable guidewires.

### BACKGROUND

Medical procedures that involve navigating to a site within the body often require a medical guidewire. Guidewires are used in numerous catheterization procedures and other medical procedures as an aid to placement of a catheter or other device at a selected site within the human body. Navigation of a guidewire often requires movement and/or steering of the guidewire through complex, tortuous anatomies to arrive at a target anatomy. A guidewire may include a steerable distal portion or tip that may be steered/articulated by a user via a controller. In order to impart such steerable behavior, the distal portion of said guidewire may be cut in various patterns to control its rigidity. This cutting process may require the use of diamond saws or lasers, which can be time-consuming and/or expensive.

### SUMMARY

This disclosure relates to, among other things, devices and methods for steerable medical devices. Each of the examples disclosed herein may include one or more of the features described in connection with any of the other disclosed examples.

According to one aspect, a medical device may comprise a handle including a handle body, and an actuator; and a shaft extending distally from the handle body. The shaft may comprise a shape memory material. The shaft may include a proximal section and a distal section The proximal section may include a first austenitic finish temperature and the distal section having a second austenitic finish temperature. The first austenitic finish temperature may be lower than the second austenitic finish temperature.

In another aspect, the shaft may further include a transition section between the proximal section and the distal section. The transition section may include a proximal portion having an austenitic finish temperature approximate to or greater than the first austenitic finish temperature. The transition section may further include a distal portion having an austenitic finish temperature approximate to or less than second austenitic finish temperature.

In another aspect, the first austenitic finish temperature may be less than or equal to body temperature, and the second austenitic finish temperature may be greater than body temperature.

In another aspect, the distal section may include at least one indentation. The at least one indentation may be a concave cut, and the at least one indentation may span at least a portion of the circumference of the distal section. The at least one indentation may span a whole circumference of the distal section. The distal section may include a bendable portion. The bendable portion may be of a lesser circumference than a circumference of the remaining portion of the distal section, and the bendable portion may be a portion of the distal section having the thinnest profile.

In another aspect, the distal section may include a plurality of indentations. The distal section may include a first indentation and a second indentation. The first indentation and the second indentation may be of different shapes, and a bending profile of the first indentation may be different from a bending profile of the second indentation. The first indentation may be a concave cut and the second indentation may be a V-shaped cut. Each of the first indentation and the second indentation may span a circumference of the distal section between approximately 20° and approximately 170°. The at least one indentation may be laser cut.

In another aspect, the medical device may further comprise a plurality of steering elements, and each of the plurality of steering elements may extend between the actuator and the distal section through an internal portion of the shaft

In another aspect, both the first austenitic finish temperature and the second austenitic finish temperature may be set via heat treatment of the proximal section and the distal section of the shaft.

According to another aspect, a medical device may comprise a shaft extending distally from the handle body. The shaft may include a shape memory material. The shaft may include a proximal section and a distal section. The proximal section may include a first austenitic finish temperature and the distal section having a second austenitic finish temperature, wherein the first austenitic finish temperature is lower than the second austenitic finish temperature, and wherein the distal section includes a first indentation defining a first bending profile and a second indentation defining a second bending profile, wherein the first bending profile is different from the second bending profile. The first bending profile may be a gradual, U-shaped bend, and the second bending profile may be an abrupt, V-shaped bend. Each of the first indentation and the second indentation may span a circumference of the distal section between approximately 20° and approximately 170°. The first austenitic finish temperature may be less than or equal to body temperature, and the second austenitic finish temperature may be greater than body temperature.

According to another aspect, a method of using a medical device, the medical device may include a handle and a shaft, wherein the handle may include an actuator, wherein the shaft may include a distal section, wherein the distal section may include a first indentation and a second indentation, and wherein the actuator may be coupled to the distal section via at least one steering element, may include delivering the shaft into a bodily lumen; positioning a distal section of the shaft adjacent to a target site; actuating the actuator so the distal section may bend about the first indentation in a first bending profile; and actuating the actuator so the distal section may bend about the second indentation in a second bending profile, wherein the first bending profile may be different from the second bending profile.

It may be understood that both the foregoing general description and the following detailed description are exemplary and explanatory only and are not restrictive of the invention, as claimed. As used herein, the terms “comprises,” “comprising,” “having,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “approximately,” or like terms (e.g., “substantially”), includes values+/−10% of a stated value.

### BRIEF DESCRIPTION OF THE FIGURES

The accompanying drawings, which are incorporated in and constitute a part of this specification, illustrate various exemplary embodiments and together with the description, serve to explain the principles of the disclosure.

FIG. 1 illustrates a side view of an exemplary medical device.

FIG. 2 depicts a side view of a distal portion of the exemplary medical device of FIG. 1.

FIG. 3 illustrates a cross-sectional view of a distal portion of the exemplary medical device of FIG. 1.

FIG. 4 illustrates a cross-sectional view of a distal portion of another exemplary medical device.

FIG. 5 illustrates a flow chart of operating the exemplary medical devices of FIGS. 1 and 4.

### DETAILED DESCRIPTION

Embodiments of this disclosure seek to provide a medical device including a steerable shaft, e.g., a guidewire. The guidewires of this disclosure may be more easily manufactured at a lesser cost relative to existing guidewires including a plurality of cuts staggered along a length of the guidewire, for example, in a diamond cut pattern. The guidewires of this disclosure may comprise a shape memory material, e.g., Nitinol, which has been subjected to a heat treatment or heat cycle process. The application of heat to said shape memory material may impart various characteristics, e.g., superelasticity and/or malleability, to heated aspects of the guidewire, thereby providing variable stiffness throughout a length of the guidewire.

A shape memory material may transition between at least two states, a martensitic state and an austenitic state. Specifically, the material transitions from an austenitic state to a martensitic state with a decrease in temperature to which the material is exposed, and similarly transitions from the martensitic state to the austenitic state with an increase in temperature to which the material is exposed. The temperature at which the transition from the austenitic state to the martensitic state begins is typically designated Ms (martensitic start temperature), while the temperature at which the transition finishes is typically designated Mf (martensitic finish temperature). As the material transitions from the austenitic state to the martensitic state, the material becomes more easily deformable. In the martensitic state, the material is able to accommodate significant deformation at low stress levels. The material exhibits malleable behavior. Due to such malleability, the material may be less likely to break as the material flexes and deforms when forces are applied to it. Thus, the material may accommodate a significant amount of deformation before reaching a breaking point.

Upon heating the material in the martensitic state, the material begins to transition to an austenitic state. The temperature at which this transition begins to occur is designated As (austenitic start temperature). The transition is complete at a temperature designated Af (austenitic finish temperature). The material may continue to accommodate deformation and exhibit malleable behavior before reaching Af. However, once the material is heated to Af, the mechanical characteristics of the material changes, and the material exhibits superelasticity, which may be defined as the ability of the shape memory material to recover from deformation. Thus, a shape memory material heated to Af may exhibit a higher degree of stiffness, pushability, and column strength relative to a shape memory material at temperatures below Af.

The Af of a shape memory material may be altered and/or set to a specific temperature by a heat treatment or heat cycling process. Thus, in some examples, a guidewire comprising a shape memory material may be heat treated, so that a particular Af (e.g., body temperature, temperatures above body temperature, etc.) may be set for the guidewire. In some other examples, various portions of the guidewire may be subjected to different heat treatments, so that each of the various portions have different Afs, resulting in variable stiffness throughout a length of the guidewire. Said heat treatment or heat cycling process for setting Af is not particularly limited, and may be any suitable heat treatment or heat cycling process known in the art.

Although the disclosure may reference guidewires used in conjunction with endoscopic, colonoscopic, and/or ERCP procedures, it will be appreciated that the guidewires may also be utilized for other types of procedures, including intracardiac, coronary vascular, central circulatory system, peripheral vascular, and neurovascular or urologic procedures. The medical devices disclosed herein may have a handle coupled to a proximal end of a shaft, e.g., a guidewire. The shaft may have at least two degrees of freedom for movement. The shaft may be articulated (e.g., bent or deflected) in one or more directions. Furthermore, the shaft may be rotatable and movable proximally and distally. The handle may be removable from the shaft, which may facilitate use of the guidewire to guide tools or medical devices after the guidewire is positioned. The shaft may include a lumen with one or two articulation wires, either or both of which may be utilized to achieve a desired steering/articulation of the shaft. The medical devices disclosed herein may be used in conjunction with duoendoscopes, endoscopes, colonoscopes, ureteroscopes, gastroscopes, bronchoscopes, laparoscopes, sheaths, catheters, or any other suitable delivery device. Moreover, the disclosed medical devices may be compatible with robotics platforms.

Reference will now be made in detail to examples of the disclosure described above and illustrated in the accompanying drawings. Wherever possible, the same reference numbers will be used throughout the drawings to refer to the same or like parts.

The terms “proximal” and “distal” are used herein to refer to the relative positions of the components of an exemplary medical device. When used herein, “proximal” refers to a position relatively closer to an operator using the medical device. In contrast, “distal” refers to a position relatively farther away from the operator using the medical device.

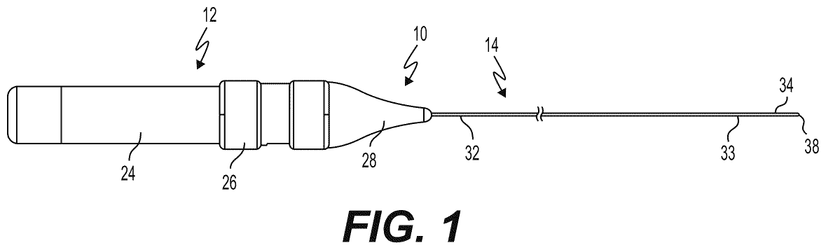

FIG. 1 depicts an exemplary medical device 10. As shown in FIG. 1, medical device 10 may include a handle 12 and a shaft 14. Handle 12 is not particularly limited and may be any suitable handle capable of steering or controlling various aspects of shaft 14. For example, handle 12 may include at least a body 24 (a handle body), an actuator 26, and a collet 28. As shown in FIG. 1, body 24 extends longitudinally. However, body 24 is not particularly limited and may be of any suitable shape or size. Portions of body 24 unencumbered by actuator 26 may be held within a hand of a user. Body 24 may include additional features or aspects (not shown) that engages and holds actuator 26 and collet 28.

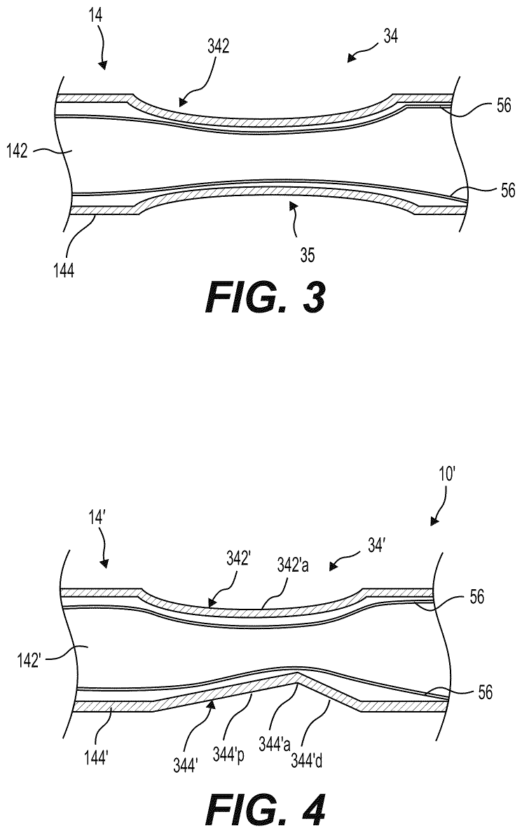

Actuator 26 may be any suitable actuator (e.g., spool, slider, knob, etc.) that is coupled to and/or movable relative to a portion of body 24. Actuator 26 may be actuated via any suitable means. For example, actuator 26 may be rotatable in a clockwise or counter-clockwise direction relative to body 24. In another example, actuator 26 may slidably translate along body 24 in a proximal and/or distal direction. Actuator 26 may be coupled to one or more steering element(s) 56 (shown in FIG. 3). For example, an aspect of actuator 26 may be coupled to proximal ends of steering element(s) 56, for example, within an internal passage of body 24.

Actuator 26 may be coupled to proximal ends of steering element(s) 56, for example, via a friction fit, an adhesive, a press fit, a crimping, or any other appropriate coupling mechanism. In this aspect, movement of actuator 26 relative to body 24 may control the pulling of one or more steering elements 56, and thus control the steering of a distal portion 34 of shaft 14. The number of steering elements 56 is not particularly limited (e.g., one or more steering elements 56), and the number of steering elements 56 may correlate with the number of different directions that shaft 14 may articulate. Moreover, although not shown, handle 12 may further include one or more springs or biasing elements, for example, within the internal lumen of body 24, to bias the movement actuator 26. For example, the one or more springs or biasing elements may bias actuator 26 to a default position in which no forces are applied to any one of steering elements 56, thereby maintaining distal portion 34 in a neutral, non-bent configuration. Additionally or alternatively, one or more portions of handle 12 may include a locking mechanism, for example, to selectively and/or releasably secure a position of actuator 26.

Collet 28 may be positioned at a distal end of body 24. As shown in FIG. 1, collet 28 may have a conical shape that tapers distally, but is not necessarily limited thereto. Collet 28 may include a distal opening (not shown) that receives and holds a proximal portion of shaft 14. The manner in which collet 28 holds shaft 14 is not particularly limited (e.g., frictional fit, etc.) Collet 28 may removably hold the proximal portion of shaft 14 so that a user, after delivery of shaft 14 to the target anatomy, may remove or uncouple handle 12 from shaft 14 via any suitable means.

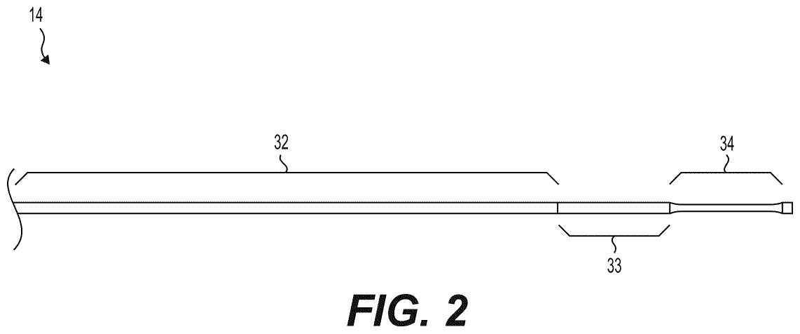

Shaft 14 may be a wire or tube-like structure (e.g., a guidewire) including an annular body 144 defining a lumen 142 (shown in FIG. 3). Shaft 14 may have any suitable outer width (e.g., diameter). For example, an outer width (e.g., diameter) of shaft 14 may be similar to or the same as a typical guidewire. Exemplary outer diameters may range from approximately 0.010 inches to approximately 0.04 inches or more particularly from approximately 0.014 inches to approximately 0.032 inches. An outer diameter may be constant along a length of shaft 14 or may vary, as shown in FIGS. 2 and 3, and discussed in further detail below.

Shaft 14 may include a proximal section 32, a transition section 33, and a distal section 34, as shown in FIGS. 1 and 2. Furthermore, shaft 14 may include a distal end 38 which is not particularly limited, and in some examples, may include a radio opaque marker visible under imaging, e.g., x-ray. A proximal portion of proximal section 32 may engage and be held within collet 28. Distal section 34 may be coupled to distal ends of steering element(s) 56 (FIG. 3), for example, via a friction fit, an adhesive, a press fit, a crimping, or any other appropriate coupling mechanism. In this aspect, the manipulation (e.g., proximal retraction and/or pulling) of steering elements 56, via actuator 26, may control the articulation or steering of distal section 34 of shaft 14.

Each of sections 32, 33, 34 of shaft 14 may comprise Nitinol and/or any other shape memory material. Moreover, as discussed above, proximal section 32 and distal section 34 may each undergo a different heat treatment or heat cycling process so that a different Af is set for each of sections 32, 34. For example, proximal section 32 may undergo a first heat treatment so that the Af of proximal section 32 is set to a first temperature (e.g., body temperature (approximately 36.2° C. to approximately 37.2° C.) or a temperature below body temperature). Distal section 34 may also undergo a second heat treatment so that the Af of distal section 34 is set to a second temperature (e.g., a temperature above body temperature). The order of the first heat treatment and the second heat treatment are not particularly limited.

As a result of the different heat treatments, proximal section 32 and distal section 34 may exhibit different mechanical characteristics when both proximal section 32 and distal section 34 are heated to a first temperature or to a second temperature, but not the other. For example, when said first temperature (e.g., Af of proximal section 32) is body temperature or a temperature below body temperature, proximal section 32 may exhibit superelasticity when inserted into a body and exposed to body temperature. Such superelasticity may help to provide proximal section 32 with improved pushability and column strength so that shaft 14 may be manipulated and/or advanced through tortuous anatomies with a reduced likelihood of buckling or deformation to proximal section 32. When said second temperature (Af of distal section 34) is a temperature above body temperature, distal section 34 maintains its malleable characteristics while inserted into the body and exposed to body temperature. Thus, distal section 34 of shaft 14 may be steerable via steering elements 56 and actuator 26.

As shown in FIGS. 1 and 2, transition section 33 may be positioned between proximal section 32 and distal section 34. Transition section 33 may be a portion of shaft 14 exposed to both a heat treatment of proximal section 32 and a heat treatment of distal section 34. As a result of such exposure, transition section 33 may include distal portions having an Af approximate to or less than the second temperature (i.e., Af of distal section 34), and proximal portions having an Af approximate to or greater than the first temperature (i.e., Af of proximal section 32). Thus, the Af of transition section 33 may gradually change throughout its length and may, for example, gradually increase between the first temperature and the second temperature, thereby exhibiting a combination of properties when heated to a certain temperature. Such variation in Af across the length of transition section 33 may help minimize kinking of transition section 33 and improve the articulation radius of transition section 33. However, in other examples, shaft 14 may be without transition section 33, and may only consist of proximal section 32 and distal section 34.

Distal section 34 may additionally include a cut or indentation (e.g., a laser cut), which may help to further improve the steerability of distal section 34. For example, as shown in FIGS. 2 and 3, distal section 34 may include an indentation 342 in body 144 such that lumen 142 tapers radially inwards throughout indentation 342. Indentation 342 may be a concave cut (i.e., extending radially inwards) around at least a portion of the circumference of distal section 34 (e.g., the whole circumference), thereby defining a bendable portion 35 that has a thinner profile (i.e., a reduced circumference) relative to a remainder of distal section 34. In some examples, bendable portion 35 may be the portion of indentation 342 having the thinnest profile relative to a reminder of distal section 34, but is not limited thereto. Bendable portion 35 may have a rigidity less than (or a greater flexibility) than the other portions of distal section 34. However, the shape and dimensions of indentation 342 are not particularly limited. Bendable portion 35 may include a consistent curvature as shown in FIGS. 2 and 3, or may include other shapes, for example, with varying proximal and distal curvatures. For example, bendable portion 35 may include a combination of proximal curvature(s), distal curvature(s), and planar sections around a portion of the circumference of distal section 34 or an entirety of the circumference of distal section 34. The manner in which indentation 342 is formed is also not particularly limited, and may be via laser cutting, sawing, grinding, filing, etc. Due to the thinner profile, bendable portion 35 may more easily bend in various directions when steering elements 56 are manipulated via actuator 26. As noted above, the number of different directions in which bendable portion 35 may articulate may correlate with the number of steering elements 56 extending from actuator 26 and coupled to distal section 34.

Heat treatment of distal section 34 including indentation 342 may help to provide improved articulation thereof (e.g., an increased degree of articulation from one side of section 34 to the other), as well as improved stress concentration preventing breakage. Such a process of heat treating distal section 34 may be more cost-effective than conventional articulation sections, which require a plurality of steering wires and corresponding lumens. Furthermore, the stiffness of the material at the flexible distal section of conventional articulation sections is the same as that of the bulk material proximal to said flexible section. The flexibility in such devices is created through a series of small mechanical linked/interlocked mechanical elements, or through a laser cut pattern that enables the laser cut portion to bend in multiple directions. In contrast, as discussed above, distal section 34 may be subjected to selective heat treatment(s) to create malleability in the shape memory material itself. Moreover, in some embodiments, additional mechanical flexibility, as well as bending properties/biases, may be imparted by the thinning of distal section 34 thereby defining indentations, e.g., indentation 342.

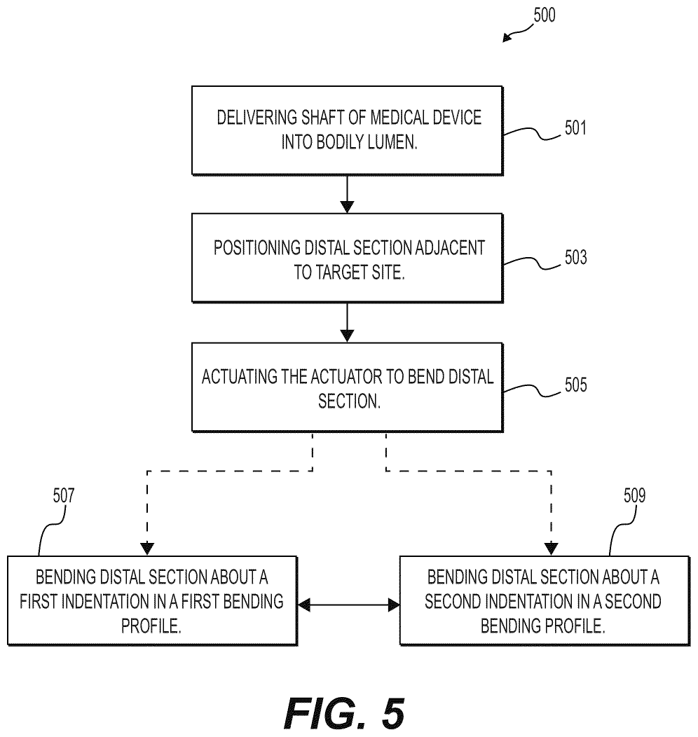

Also referring to FIG. 5, an exemplary method 500 of how medical device 10 may be used is further discussed. A user may deliver a distal end of a shaft of a mother scope (e.g., an endoscope) into the body of a subject, for example, via a natural orifice (such as a mouth or anus). The distal end of the shaft of the motherscope may be delivered through a tortuous natural body lumen of the subject, such as an esophagus, stomach, colon, etc., towards an intended target site, as described in step 501. The user may then deliver shaft 14 (e.g., a guidewire) through a channel or lumen of the shaft of the mother scope towards or adjacent to the intended target site, as described in step 503. At least a portion of distal section 34 may extend distally of the mother scope. As shaft 14 is delivered into the body, proximal section 32 may be heated by the subject's body to its Af, which may be set to body temperature or a temperature below body temperature, thereby transitioning proximal section 32 to a superelastic state. Meanwhile, distal section 34 may maintain its malleable state, as distal section 34 may have an Af above body temperature. The user may then actuate actuator 26 of handle 12 to articulate or bend distal section 34 of shaft 14, as described in step 505, so that distal end 38 of shaft 14 may be adjacent to the intended target site.

FIG. 4 depicts a portion of another exemplary shaft 14′ of a medical device 10′. Shaft 14′ may having a plurality of different bending profiles. Distal section 34′ of shaft 14′ may include two different cuts or indentations 342′, 344′ in body 144′ so that lumen 142′ tapers radially inwards throughout indentation the portion of distal section 34′ including indentations 342′, 344′. Each of indentations 342′, 344′ may impart a different bending profile to shaft 14′. However, the number of different cuts or indentations is not particularly limited, and other exemplary shafts may include three, four, five, six, etc. different cuts or indentations, yielding a plurality of different bending profiles.

First indentation 342′ may be a concave, semi-circular cut (i.e., extending radially inwards) around a partial circumference of distal section 34. The shape and dimensions of first indentation 342′ are not particularly limited. The degree by which first indentation 342′ extends around or spans a circumference of distal section 34′ is also not particularly limited, and may, for example, range between approximately 20° and approximately 170°. First indentation 342′ yields a first bending profile of distal section 34′. Given the semi-circular cut of first indentation 342′, the first bending profile may be a gradual, U-shaped bend around a central point 342′a, and may also be referred to as a ‘sugar-cane’ bending profile. Such a bending profile may define a gradual curvature around central point 342′a, and the bending profile may be similar in shape to that of a sugar cane, candy cane, and other similar shapes. The degree of bend around point 342′a that may be possible is dependent on the shape and dimensions of first indentation 342′.

Second indentation 344′ may be a V-shaped cut (i.e., extending radially inwards) around a partial circumference of distal section 34, for example, around a portion of the circumference of distal section 34 that is unencumbered by first indentation 342′. The shape and dimensions of second indentation 344′ are also not particularly limited. The degree by which second indentation 344′ extends around or spans a circumference of distal section 34′ is also not particularly limited, and may, for example, range between approximately 20° and approximately 170°. In some examples, second indentation 344′ may extend around distal section 34′ by the same degree in which first indentation 342′ extends around distal section 34′. However, the degree by which second indentation 344′ extends around distal section 34′ may also differ from that of first indentation 342′. Second indentation 344′ yields a second bending profile of distal section 34′. Given the V-shaped cut of second indentation 344′, the second bending profile of distal section 34′ may bend about low point 344′a, and may also be referred to as a ‘hockey stick’ bending profile. Second indentation 344′ may include a proximal portion 344′p and a distal portion 344′d on both sides of low point 344′a. As shown in FIG. 4, the V-shaped cut of second indentation 344′ may be asymmetrical so that the angle of proximal portion 344′p relative to a longitudinal axis of shaft 14′ is different from the angle of distal portion 344′d relative to the longitudinal axis of shaft 14′. However, in other examples, the V-shaped cut of second indentation may be symmetrical so that the angle of proximal portion 344′p relative to the longitudinal axis of shaft 14′ is the same as the angle of distal portion 344′d relative to the longitudinal axis of shaft 14′. The bend about low point 344′a may be sharper or more abrupt compared to the bend about central point 342′a of first indentation 342′. The degree of bend that may be feasible via second indentation 344′ may be dependent on the shape and dimensions of second indentation 344′.

Medical device 10′ may be used in the same or similar manner as discussed above for medical device 10. Additionally, referring to FIG. 5, the user may manually rotate shaft 14′ (e.g., with a handle similar to handle 12) to utilize either the first bending profile of first indentation 342′ or the second bending profile of second indentation 344′ in any direction via pulling of steering elements 56. The user may then may then actuate actuator 26 of handle 12 to articulate or bend distal section 34′ about first indentation 342′ in a first bending profile, as described in step 507, and/or articulate or bend distal section 34′ about second indentation 344′ in a second bending profile, as described in step 509.

It is contemplated that the guidewires and methods discussed herein may be applicable to any endoscopic and/or minimally invasive procedure. For example, the systems, devices, and methods discussed above may be used during a percutaneous nephrolithotomy/nephrolithotripsy (PCNL), endoscopic retrograde cholangiopancreatography (ERCP), balloon and laser angioplasty, nephrostomy, electrode placement, etc. The systems, devices, and methods discussed above may also be used in procedures to remove ureteral stones, gallstones, bile duct stones, polyps, stent placement, gastroenteral anastomosis, choledochoduodenostomy, etc. The systems, devices, and methods discussed above may also be used in intracardiac, coronary vascular, central circulatory system, peripheral vascular, and neurovascular procedures.

While principles of the disclosure are described herein with reference to illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Accordingly, the claimed features are not to be considered as limited by the foregoing description.

We claim:

1. A medical device comprising:
a handle including:
a handle body, and
an actuator; and
a shaft extending distally from the handle body, wherein the shaft comprises of a shape memory material,
wherein the shaft includes a proximal section and a distal section, the proximal section having a first austenitic finish temperature and the distal section having a second austenitic finish temperature, and
wherein the first austenitic finish temperature is lower than the second austenitic finish temperature.

2. The medical device of claim 1, wherein the shaft further includes a transition section between the proximal section and the distal section, wherein the transition section includes a proximal portion having an austenitic finish temperature approximate to or greater than the first austenitic finish temperature, and wherein the transition section further includes a distal portion having an austenitic finish temperature approximate to or less than second austenitic finish temperature.

3. The medical device of claim 1, wherein the first austenitic finish temperature is less than or equal to body temperature, and wherein the second austenitic finish temperature is greater than body temperature.

4. The medical device of claim 1, wherein the distal section includes at least one indentation.

5. The medical device of claim 4, wherein the at least one indentation is a concave cut, and the at least one indentation spans at least a portion of a circumference of the distal section.

6. The medical device of claim 5, wherein the at least one indentation spans a whole circumference of the distal section.

7. The medical device of claim 4, wherein the distal section includes a bendable portion, wherein the bendable portion is of a lesser circumference than a circumference of a remaining portion of the distal section, and wherein the bendable portion is a portion of the distal section having the thinnest profile.

8. The medical device of claim 4, wherein the distal section includes a plurality of indentations.

9. The medical device of claim 4, wherein the distal section includes a first indentation and a second indentation.

10. The medical device of claim 9, wherein the first indentation and the second indentation are of different shapes, and a bending profile of the first indentation is different from a bending profile of the second indentation.

11. The medical device of claim 10, wherein the first indentation is a concave cut and the second indentation is a V-shaped cut.

12. The medical device of claim 10, wherein each of the first indentation and the second indentation spans a circumference of the distal section between approximately 20° and approximately 170°.

13. The medical device of claim 4, wherein the at least one indentation is laser cut.

14. The medical device of claim 1, further comprising a plurality of steering elements, wherein each of the plurality of steering elements extends between the actuator and the distal section through an internal portion of the shaft.

15. The medical device of claim 1, wherein both the first austenitic finish temperature and the second austenitic finish temperature are set via heat treatment of the proximal section and the distal section of the shaft.

16. A medical device comprising:
a shaft extending distally from a handle body, wherein the shaft comprises of a shape memory material,
wherein the shaft includes a proximal section and a distal section, the proximal section having a first austenitic finish temperature and the distal section having a second austenitic finish temperature,
wherein the first austenitic finish temperature is lower than the second austenitic finish temperature, and
wherein the distal section includes a first indentation defining a first bending profile and a second indentation defining a second bending profile, wherein the first bending profile is different from the second bending profile.

17. The medical device of claim 16, wherein the first bending profile is a U-shaped bend, and wherein the second bending profile is a V-shaped bend.

18. The medical device of claim 16, wherein each of the first indentation and the second indentation spans a circumference of the distal section between approximately 20° and approximately 170°.

19. The medical device of claim 16, wherein the first austenitic finish temperature is less than or equal to body temperature, and wherein the second austenitic finish temperature is greater than body temperature.

20. A method of using a medical device, the medical device including a handle and a shaft, wherein the handle includes an actuator, wherein the shaft includes a distal section, wherein the distal section includes a first indentation and a second indentation, and wherein the actuator is coupled to the distal section via at least one steering element, the method comprising:
delivering the shaft into a bodily lumen;
positioning the distal section of the shaft adjacent to a target site;
actuating the actuator to bend the distal section about the first indentation in a first bending profile; and
actuating the actuator to bend the distal section about the second indentation in a second bending profile,
wherein the first bending profile is different from the second bending profile.
